(19) United States
(12) Patent Application Publication
(10) Pub. No.: US 2009/0074668 A1
(43) Pub. Date: Mar. 19, 2009

(54) Vldlr-/- mouse models and related methods

(76) Inventor: Rafal A. Farjo, Oklahoma City, OK (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/283,393

(22) Filed: Sep. 11, 2008

Related U.S. Application Data
(60) Provisional application No. 60/972,743, filed on Sep. 14, 2007.

(51) Int. Cl. A61K 49/00

(52) U.S. Cl. 424/9.2

(57) ABSTRACT

A Vldlr−/− mouse model and related methods has been shown to be an effective model for eye-disease studies and determination of effective therapeutics. Vldlr−/− mice are observed having both chorodial neovascularization coupled with subretinal deposits and photoreceptor atrophy. The mice of the present disclosure have knocked out the very low-density lipoprotein receptors. Similarly, methods of determination of effective therapeutics for age-related macular degeneration are disclosed herein.

### RELATED APPLICATION

This application claims the benefit of and priority to U.S. Provisional Application Ser. No. 60/972,743, filed Sep. 14, 2007, the contents of which are incorporated by reference herein in their entirety.

### BACKGROUND

This disclosure relates to methods for the screening agents useful in treating eye-related diseases, such as age-related macular degeneration, using a mouse model with cells deficient in the very low-density lipoprotein receptor gene.

### SUMMARY

A Vldlr−/− mouse model and related methods has been shown to be an effective model for eye-disease studies and determination of effective therapeutics. Vldlr−/− mice are observed having both chorodial neovascularization coupled with subretinal deposits and photoreceptor atrophy. The mice of the present disclosure have knocked out the very low-density lipoprotein receptors. Similarly, methods of determination of effective therapeutics for age-related macular degeneration are disclosed herein.

According to a feature of the present disclosure, a method is disclosed comprising identifying a candidate therapeutic agent for the treatment of an eye-related diseased characterized by at least one of vascular leakage, inflammation, ocular disease characterized by over-active wnt pathway signaling, or overexpression of LRP₅ or LRP6n causing the agent to be administered to a mouse whose cells comprise at least a disrupted very low-density lipoprotein receptor, the disruption being sufficient to disrupt substantial expression of very low-density lipoprotein receptor, and causing a determination of the effectiveness of the agent in treating the eye-related disease. The mouse exhibits choroidal neovascularization, and the mouse exhibits at least one of neo-vascularization in the eye and inflammation of the eye.

According to a feature of the present disclosure, a method is disclosed comprising, identifying a candidate therapeutic agent for the treatment of age-related macular degeneration, causing the agent to be administered to a mouse whose cells comprise at least a disrupted very low-density lipoprotein receptor, the disruption being sufficient to disrupt substantial expression of very low-density lipoprotein receptor, and causing a determination of the effectiveness of the agent in treating the age-related macular degeneration. The mouse exhibits choroidal neovascularization, as well as at least one of neo-vascularization in the eye and inflammation of the eye.

### DRAWINGS

The patent or application file contains at least one drawing executed in color. Copies of this patent application publication with color drawing(s) will be provided by the Office upon request and payment of the necessary fee.

The above-mentioned features and objects of the present disclosure will become more apparent with reference to the following description taken in conjunction with the accompanying drawings wherein like reference numerals denote like elements and in which:

FIG. 1 are photographs of embodiments of tissue slices having neovascularization;

FIG. 2 are photographs of embodiments of eye tissue cross sections showing the lack of pericytes in Vldlr−/− mice;

FIG. 3 are photographs of embodiments of vascular leakage in Vldlr−/− mice compared to wild type mice;

FIG. 4 are experimental data and photographs of embodiments of Vldlr−/− mice showing increased VEGF and VEGFR1 in Vldlr−/− eyecups;

FIG. 5 are photographs of embodiments of Vldlr−/− mice exhibiting altered polarization of VEGF distribution in RPE cells;

FIG. 6 are photographs and a graph of embodiments of Vldlr−/− mice exhibiting increased leukostasis in the Vldlr−/− retina;

FIG. 7 is an embodiment of a western blot showing over-expression of TNF-α in Vldlr−/− eyecups;

FIG. 8 are photographs of eye cross sections showing increased nuclear translocation of NF-κB in the retina and choroid of Vldlr−/− mice;

FIG. 9 are photographs of embodiments of the fundus of the eye and eye tissue cross sections showing photoreceptor degeneration in Vldlr−/− mice;

FIG. 10 is a graph of an embodiment of disturbed retinoid profile in Vldlr−/− eyecups;

FIG. 11 is a graph of an embodiment of the decline of Cone Photoreceptor derived ERG amplitudes in Vldlr−/− mice;

FIG. 12 are photographs of embodiments of eye tissue cross sections showing increased HIF-1α expression in Vldlr−/− eyecups;

FIG. 13 are tissue slice photographs and graphs of embodiments showings progressive degeneration of photoreceptors in Vldlr−/− mice; and

FIG. 14 are photographs and graphs of embodiments of over-expression of inflammatory factors in Vldlr−/− eyecups.

### DETAILED DESCRIPTION

In the following detailed description of implementations of the present disclosure, reference is made to the accompanying drawings in which like references indicate similar elements, and in which is shown by way of illustration specific implementations in which the present disclosure may be practiced. These implementations are described in sufficient detail to enable those skilled in the art to practice the present disclosure, and it is to be understood that other implementations may be utilized and that logical, mechanical, electrical, functional, compositional, and other changes may be made without departing from the scope of the present disclosure. The following detailed description is, therefore, not to be taken in a limiting sense, and the scope of the present disclosure is defined only by the appended claims. As used in the present disclosure, the term “or” shall be understood to be defined as a logical disjunction and shall not indicate an exclusive disjunction unless expressly indicated as such or notated as “xor.”

As used herein, the term “eye-related disease” shall mean diseases of the eye characterized by at least inflammation, angiogenesis, and neovascularization.

Age-related macular degeneration is expressly contemplated as falling within the definition of “eye-related disease.” Other diseases include: ocular disease characterized by over-active wnt pathway signaling, or overexpression of LRP5 or LRP6n, diabetic retinopathy, diabetic macular edema, retinitis, and uveitis.

As used herein, the term “agent” shall mean a compound that has a beneficial effect in treating an eye-related disease.

Age-related macular degeneration (AMD) is a rapidly growing retinal disease that primarily affects patients of age 50 years and older. Current prevalence rates in the US estimate that over 1.75 million citizens are afflicted with this disorder, however, as a consequence of the rapidly growing aging population, it is predicted that the number of persons afflicted with AMD will increase 50% to 2.95 million by 2020.

There are two major classifications of AMD, dry and wet. All patients initially present with dry-AMD, which entails the accumulation of debris and deposits in the outer retina known as drusen and atrophic and hypertrophic changes in the retinal pigment epithelium (RPE). This disease may progress into the “wet” form of the disease, whereby choroidal neovascularization occurs and causes a leakage of plasma and fluid into the retina. Only 20% of all AMD patients develop the wet form of the disease; however, 90% of blindness due to AMD is caused by wet-AMD. These patients present with loss central vision that worsens with age. This causative loss of vision is largely in part an effect of the abnormal neovascularization that causes vascular leakage into the retina. The progressive vascular leakage leads to photoreceptor apoptosis and initiation of inflammatory pathways, which permanently inhibit the potential for therapeutic intervention.

The present disclosure is applicable to other eye-disease states characterized by at least one of inflammation, angiogenesis, or neovascularization. For example, the present disclosure is applicable age-related macular degeneration, diabetic retinopathy, diabetic macular edema, retinitis, and uveitis.

### VEGF is a Mediator of Retinal Vascular Leakage

It has been shown that growth factors, such as VEGF and PEDF are implicated in the pathogenesis of AMD. VEGF is a potent mediator of vascular permeability and angiogenesis and a potent mitogen with a unique specificity for endothelial cells in a variety of human pathological situations. The increased VEGF levels are responsible for the retinal vascular leakage or retinal vascular hyper-permeability.

Clinical and animal studies have shown that VEGF plays a pivotal role in the development of AMD. Current pharmacological treatments for AMD, including ranibizumab (Lucentis®) and pegaptanib (Macugen®) are both inhibitors that bind VEGF and prevent subsequent initiation of pathways leading to neovascularization. Kenalog is another proposed treatment for macular edema and the prevention of neovascularization. Intraocular administration of Kenalog has been shown to reduce VEGF levels in vitro and in vivo; however, Kenalog is a steroid with potentially serious side effects in human patients. The only other therapy for AMD is surgically based, and known as photo-dynamic therapy. This entails the systemic injection of a tracer dye into the patient, followed by cauterization of aberrant retinal angiogenesis by a laser.

Several large genetic population studies have been carried out to identify susceptibility loci contributing to AMD development. These studies have identified several loci demonstrating significant correlation to the risk of developing AMD, depending on the population studied. Based on these results, it is suggested that AMD is a complex disease that occurs as a result of several environmental and genetic disposition elements; however, these association studies have identified a number of inflammatory-related genes that predispose individuals to AMD.

In multiple populations, mutations in the complement factor H (CFH) gene have been identified that are present in a significant number of AMD afflicted individuals. CFH is present in the bloodstream and is an inhibitor of complement activation. Thus, it has been suggested that the CFH mutations observed in AMD patients may represent an inability for these individuals to restrict inflammation in the retina as a result of vascular leakage. In addition, another large association study has implicated variants of toll-like receptor 4 (TLR4) in contributing to AMD susceptibility. TLR4 has been demonstrated to play a significant role in pro-inflammatory signaling pathways, which may also contribute to the inflammation observed in AMD pathogenesis. A recent study demonstrated the significant linkage and association of VLDLR variants in AMD patients. Since this study used familial linkage analysis and association studies with case controls, the likelihood of VLDLR involvement in many forms of AMD pathogenesis is high.

It is generally difficult to create an animal model for AMD that exactly mimics all the features of AMD. Mice are typically the optimal species to use due to their quick breeding and maturation times. In the case of AMD, however, mouse models are difficult to obtain because mice do not have the large population of cone photoreceptors in their central retina (macula) as humans, and the central retina is the site of initial photoreceptor cell loss in humans. Other non-rodent species do have a macula, but the long wait for breeding and maturation makes them an inefficient model. Several genetic mouse models of AMD have been created based upon the results of human linkage and association studies and many of these display the accumulation of drusen and photoreceptor/RPE atrophy. These are useful models, but they fail to address the underlying cause of visual loss, choroidal neovascularization (CNV).

One mouse model, harboring a disruption in the monocyte chemotactic protein 1 (MCP-1) gene has demonstrated CNV coupled with subretinal deposits and photoreceptor atrophy; however, drug screening toxicity in this model is impossible since MCP-1 is a key inflammatory factor. The absence of this factor prevents the true evaluation of a potential immune response caused by the drug administered. Another non-genetic model of CNV is used whereby a laser is used to induce CNV. While this method is highly effective in producing true CNV, it is not very reproducible in biological replicates due to the variability in location and damage caused by the laser between animals.

### VLDLR Receptor Plays Role in Angiogenesis Regulation

Very low-density lipoprotein receptor (VLDLR) is a member of the low-density lipoprotein (LDL) receptor gene family. It was originally named VLDLR as it mediates binding and uptake of very low-density lipoprotein (VLDL), but not LDL. Unlike the LDL receptor, VLDLR has a widespread expression in many tissues and was suggested to play a role in lipoprotein metabolism. Later, several ligands for VLDLR have been identified, including plasminogen inhibitor type 1 (PAI-1), and endogenous anti-angiogenic factors, such as thrombospondin-1 (TSP-1) and tissue factor pathway inhibitor (TFPI). Further, VLDLR has been shown to mediate VLDL-induced PAI-1 gene transcription via regulating a transcription factor, namely VLDL-inducible factor-1. These findings suggest that VLDLR may be also coupled with intracellular signaling pathways and mediate other functions in addition to lipoprotein metabolism.

VLDLR gene knockout (Vldlr−/−) mice were initially created to study the functions of VLDLR in lipid metabolic pathways. However, Vldlr−/− mice have been shown to be viable and fertile. Under normal diet conditions, plasma levels of cholesterol, triglyceride, and lipoproteins were found normal in Vldlr−/− mice. Surprisingly, in a comprehensive ocular phenotype screen, it was discovered through fundus examination that Vldlr−/− mice develop abnormal and progressive subretinal neovascularization (NV). This surprise report suggests that VLDLR also plays a role in angiogenesis regulation. To date, the underlying mechanism or signaling pathway by which disruption of the Vldlr gene (knockout) leads to the subretinal NV has not been elucidated.

Genes Associated with Age-Related Macular Degeneration

A recent linkage and allelic association test using a family-based association dataset and an independent case control dataset revealed genetic variations in five of the genes analyzed. Among these, VLDLR, LRP6 and VEGF were found to have significant association with AMD. This study suggests potential roles of VLDLR and the wnt pathway in the development or progression of AMD.

An unmet need currently exists to develop a genetic model of AMD that can be reproducibly used to assess the effect of therapeutics. Laser-induced CNV is commonly used as an AMD model. However, the following features prevent its wide use as a model for drug screening: (1) It is a wound healing model, so that the pathogenesis is not relevant to AMD; (2) laser induced CNV varies widely, dependent on the laser dose, focus of the laser and location of the laser burn in the retina; (3) it is difficult to objectively quantify CNV; and (4) each retina needs to receive multiple spots of laser burn to avoid location-related variations in CNV. Thus, induction of CNV is labor-intensive. Therefore, a genetic, reproducible, and quantifiable CNV model in mammalians is of great significance for development of new drugs for AMD and of great market for contract service.

The inventors of the present disclosure demonstrated that Vldlr−/− mice are an AMD model with true CNV that can be used to screen new anti-angiogenic and anti-inflammatory compounds in development for the treatment of AMD. This model develops most phenotypes of human AMD such as retinal inflammation, vascular leakage, progressive photoreceptor degeneration, and CNV. More importantly, the CNV is early onset and highly reproducible and easy to quantify. The mice of the present disclosure provide an in vivo platform to screen drugs for other pharmaceutical companies.

The mouse models and methods of the present disclosure, according to embodiments, are used for screening: (1) anti-inflammatory agents, (2) anti-vascular permeability agents, (3) anti-angiogenic agents, and (4) agents preventing photoreceptor degeneration.

### EXAMPLES

1. Vldlr−/− mice develop early onset and progressive CNV

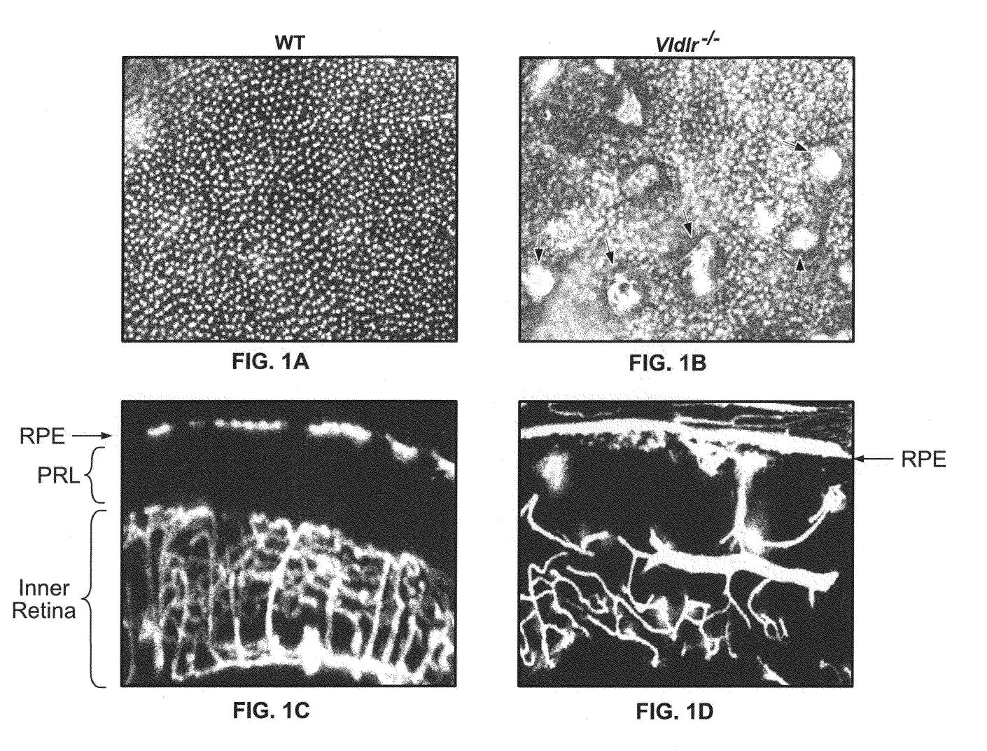

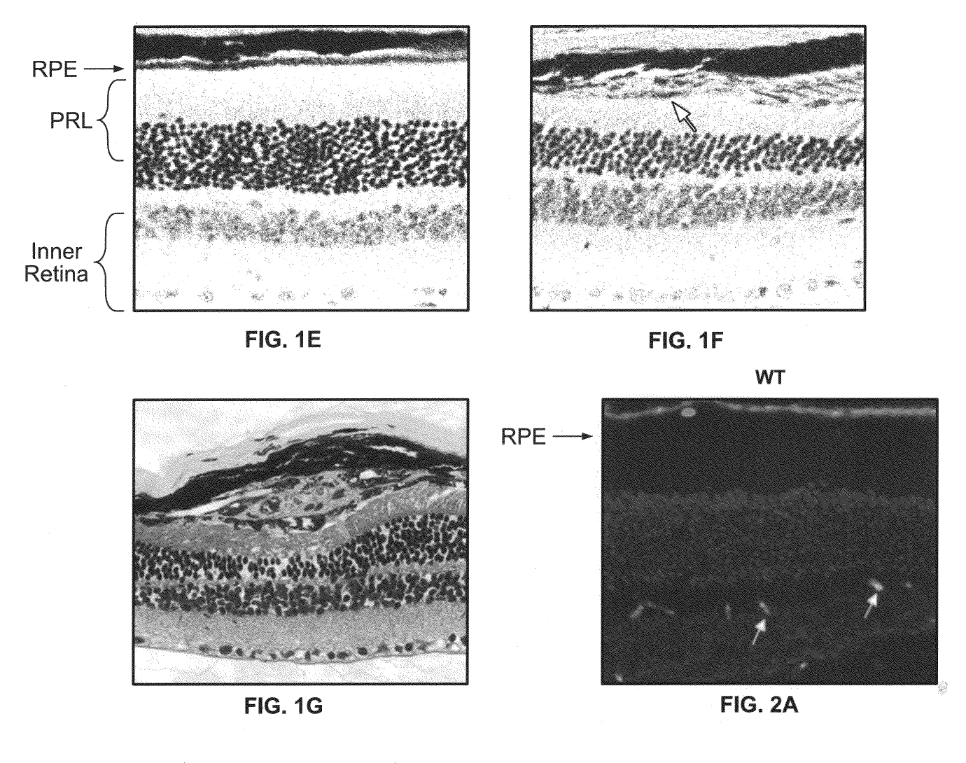

Vasculature in the retina and choroid in Vldlr−/− and wild type (wt) mice at ages of postnatal day 12 (P12) and 6 wks were examined by both in vivo vessel staining and fluorescein-angiography using high molecular weight fluorescein isothiocyanate (FITC)-conjugated dextran. In Vldlr−/− mice at P12, there is no detectable neovascularization in the retina, while choroidal neovasculature can be seen to have penetrated through Bruch's membrane and the RPE (see, e.g., FIG. 1B, arrows). This assay allows for the visualization and quantification of NV in rodent eyes. After peeling off the retina, multiple vessel penetration sites can be observed on the surface of the RPE-choroid flat mount, in contrast to the intact RPE layer in age-matched wt mice (FIG. 1A), suggesting that CNV occurs earlier than the intra-retinal NV.

At 6 wks of age, Vldlr−/− mice showed abnormal NV throughout the subretinal space and the photoreceptor layer (FIG. 1D), consistent with the previous observations. By 6 wks of age, the choroidal vascular network has anastomosed with the retinal vasculature (FIG. 1D). In older Vldlr−/− mice (7 months), vasculature accumulated in the subretinal space, a typical pathological feature in wet AMD in humans (FIGS. 1F and 1G). This systematic examination of the CNV time course suggests that this is an early onset and progressive CNV model.

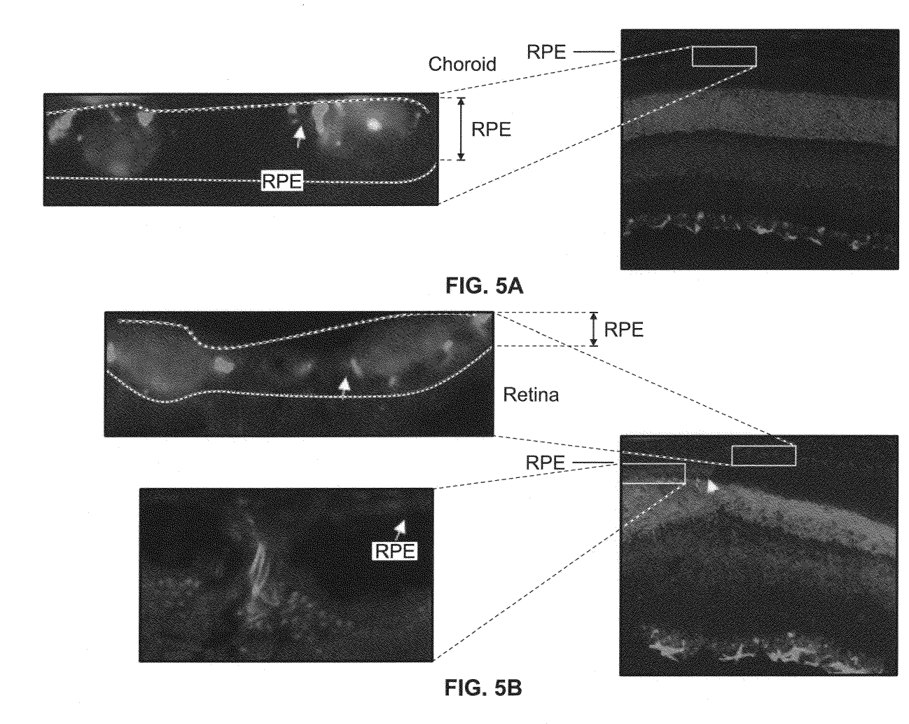

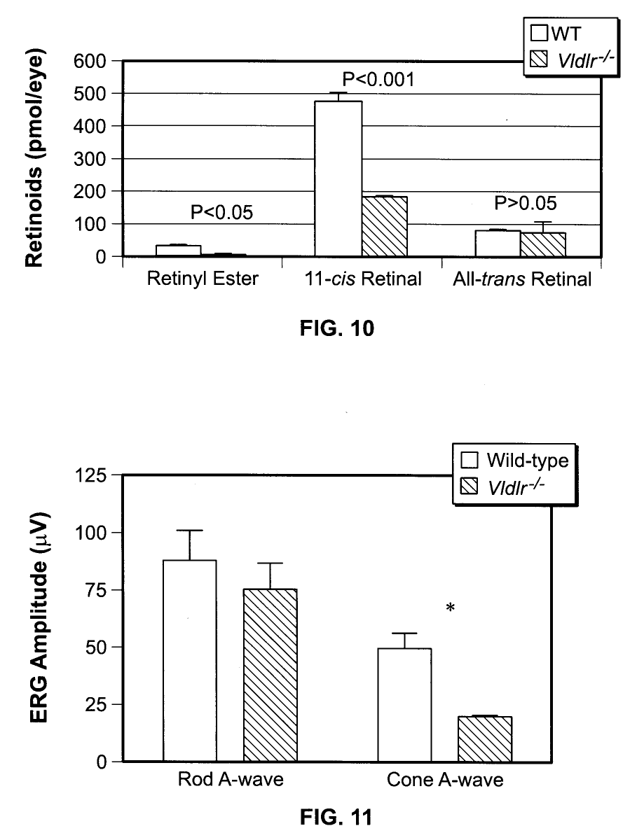

According to embodiments of experimental data and as illustrated in FIGS. 1A and 1B, choroidal-RPE are shown in flat mounts following intravascular filling with fluorescein-conjugated high molecular weight dextran at age P12. Unlike wt shown in FIG. 1A, which has an intact retinal pigment epithelium (RPE) layer, the neovasculature already penetrated from the choroid through Bruch's membrane and the RPE (large fluorescent spots shown in FIG. 1B) in Vldlr−/− mice (arrows). Note that the small dots in the RPE cell layer are due to autofluorescence of RPE cell nuclei. FIGS. 1C and 1D show thick retinal cross sections (200 μm) following in vivo vessel staining with an FITC-conjugated antibody against collagen IV to visualize the vascular network (RPE cells show some auto-fluorescence). In FIG. 1C, a fluorescein angiography shows no vasculature in the photoreceptor layer (PRL) in wt mice. In the Vldlr−/− retina shown in FIG. 1D, perpendicular vessels penetrated from the choroid to PRL. Cross ocular sections are shown in FIGS. 1E and 1F with hematoxylin and eosin (HE) staining show accumulated neovasculature in the subretinal space in aged Vldlr−/− mice (7 months of age) (FIG. 1F), but not in age-matched wt control (FIG. 1E). Note that Vldlr−/− mice have shortened rod outer segments, decreased nucleus layers of photoreceptors and retinal detachment in some areas. The arrow in FIG. 1F indicates examples of the neovasculature in the subretinal space. FIG. 1G shows a high-magnification image showing subretinal neovasculature (examples indicated by the arrow) in Vldlr−/− mice. The scale for each of FIGS. 1A-1G: 100 μm in FIGS. 1A and 1B, 20 μm in FIGS. 1C and 1D, 10 μm in FIGS. 1E and 1F, and 5 μm in FIG. 1G.

### 2. Vldlr Gene Knockout Impairs the Vascular Maturity and Integrity in the Eye

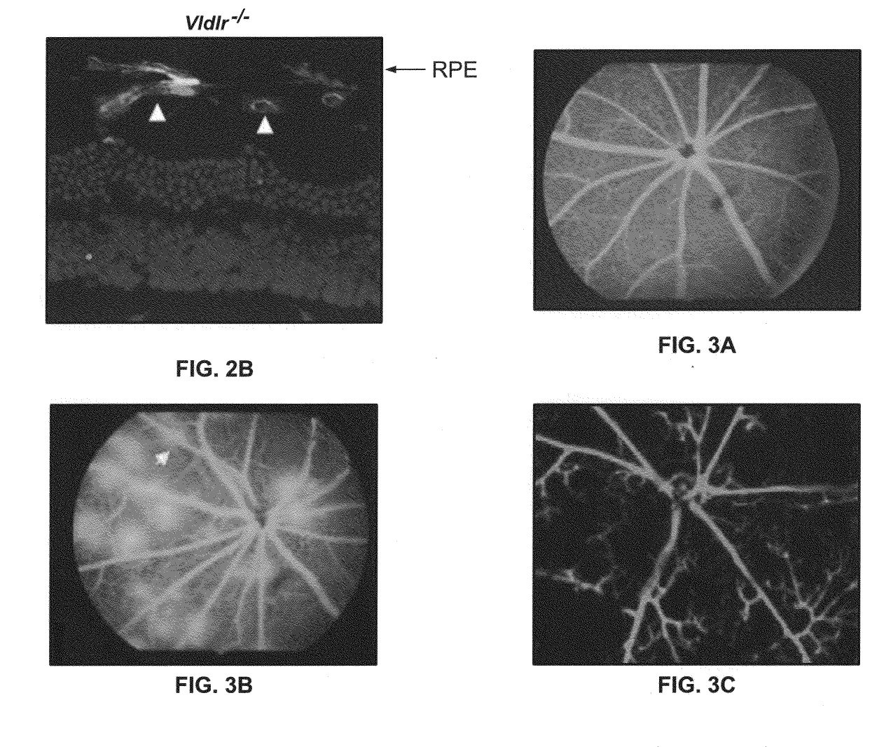

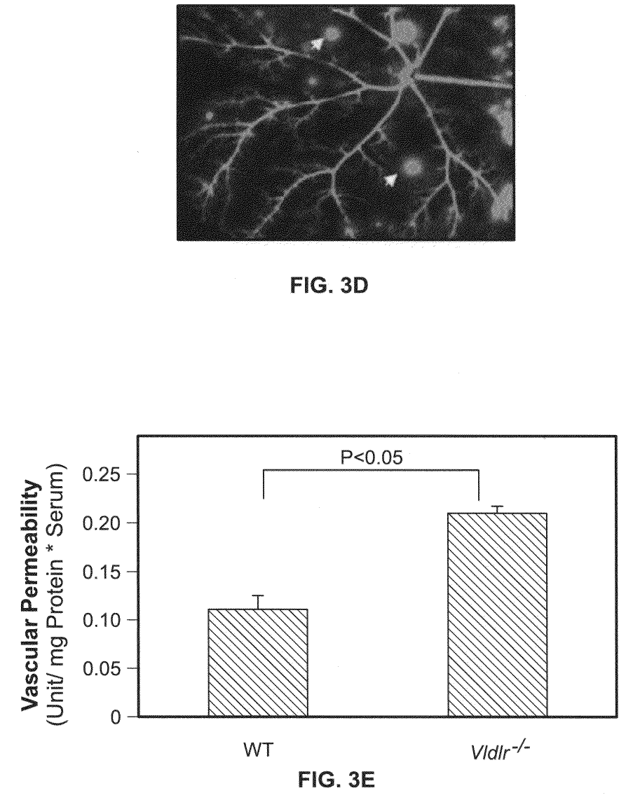

To examine the integrity and maturity of the neovasculature in the mice of the present disclosure, pericyte coverage of the capillaries was examined by double immunostaining of CD31 (an endothelial marker) and SMA (a pericyte marker). In wt mice (6 wks of age), CD31-positive endothelial cells were accompanied by SMA-positive pericytes in the inner retina, demonstrating maturity of retinal vasculature at this age. In contrast, the retinal and subretinal neovasculature in Vldlr−/− mice at the same age showed only CD31 staining, but no SMA signal, suggesting that endothelial cells are not covered by pericytes in the neovasculature in Vldlr−/− mice (FIGS. 2A and 2B). Consistent with the impaired vascular maturation, retinal capillaries displayed significant leakage in the Vldlr−/− retina as seen both in fundus images and flat-mounted retina after FITC angiography (FIGS. 3B and 3D). Quantification by vascular permeability assay confirmed that Vldlr−/− mice (age of 2 months) have significantly higher retinal vascular permeability than that in the age-matched wt mice, indicating vascular leakage in the Vldlr−/− retina (FIG. 3E).

According to embodiments of exemplary data, FIG. 2 shows the lack of pericytes in subretinal neovasculature in Vldlr−/− mice. At age of 6 wks, cross eye sections from wt (FIG. 2A) and Vldlr−/− (FIG. 2B) mice were double stained with an anti-CD31 antibody to label endothelial cells (green) and an anti-SMA antibody to label pericytes (red). In the inner retinal layer of wt mice, endothelial cells are covered by pericytes (FIG. 2A), while in the subretinal vasculature in the Vldlr−/− retina, endothelial cells are not accompanied by pericytes (exemplified by arrows FIG. 2B). For FIGS. 2A and 2B, the scale is 10 μm.

According to embodiments of exemplary data, FIG. 3 shows retinal vascular leakage in Vldlr−/− mice. In FIGS. 3A and 3B, FITC-dextran (molecular weight of 2000 kDa) was injected into the left ventricle. The fundus image was taken 3 min after the injection in wt (FIG. 3A) and Vldlr−/− (FIG. 3B) mice. After an intravenous injection of FITC-BSA, the retina was dissected, fixed immediately, and flat-mounted in FIGS. 3C and 3D. Retinal vasculature was visualized under a fluorescent microscope. Leakage of FITC-dextran or FITC-BSA from retinal vasculature was observed in the Vldlr−/− retina in both the fundus images and in the flat-mounted retinas (FIGS. 3B and 3D, respectively), but not in the wt retina (FIGS. 3A and 3C). Retinal vascular permeability was quantified using the FITC-BSA injected into the tail vein as a tracer as shown in FIG. 3E, and FITC-BSA leaked to the retina was measured by a fluorometer and normalized by total protein concentrations in the retina and serum FITC levels (mean±SD, n=6).

### 3. Over-Expression of VEGF and VEGFR2 in Vldlr−/− Eyecups

As VEGF is widely considered the major angiogenic factor in the retina, we compared VEGF expression in the eyecups of Vldlr−/− mice with those of the age-matched wt mice at both the protein and mRNA levels. Western blot analysis demonstrated significantly elevated levels of the VEGF monomer and dimer in Vldlr−/− eyecups in comparison to the wt eyecups. Real-time RT-PCR showed that the increased VEGF expression occurs at the mRNA level in the Vldlr−/− mice. Moreover, VEGFR2 levels were also elevated in the Vldlr−/− eyecups. To provide additional evidence for the causative role of VEGF over-expression in the subretinal NV in Vldlr−/− mice, we injected neutralizing antibodies (10 μg/eye) specific for VEGFR2 and VEGFR3 separately into the subretinal space of Vldlr−/− mice at age of P12. Immunostaining of CD31, an endothelial marker at age of 6 wks demonstrated that an injection of the anti-VEGFR2 antibody abrogated the subretinal NV, while the identical dose of the anti-VEGFR3 antibody had no effect on subretinal NV, suggesting the role of VEGFR2 in mediating the angiogenic effect of VEGF in this mouse model.

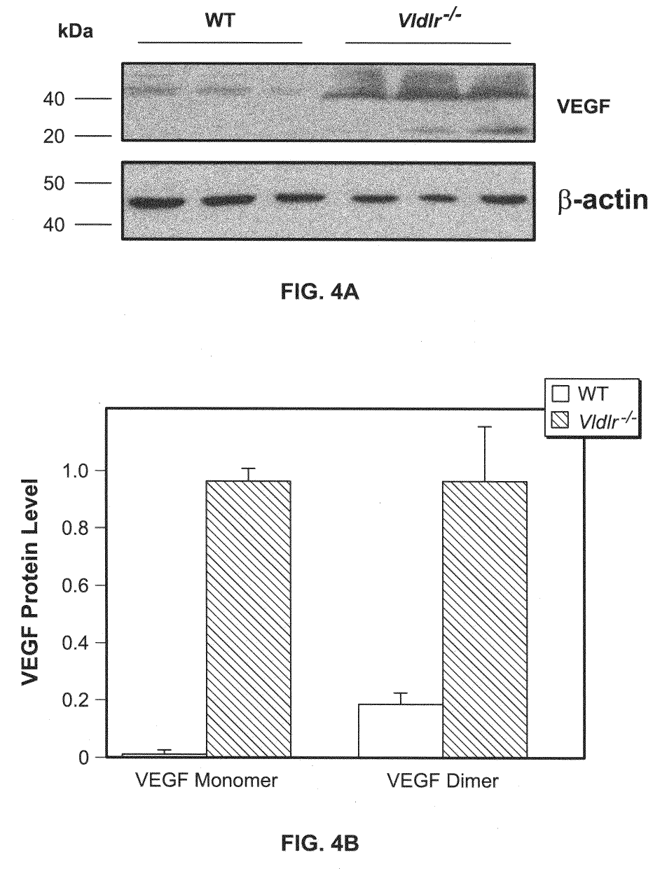

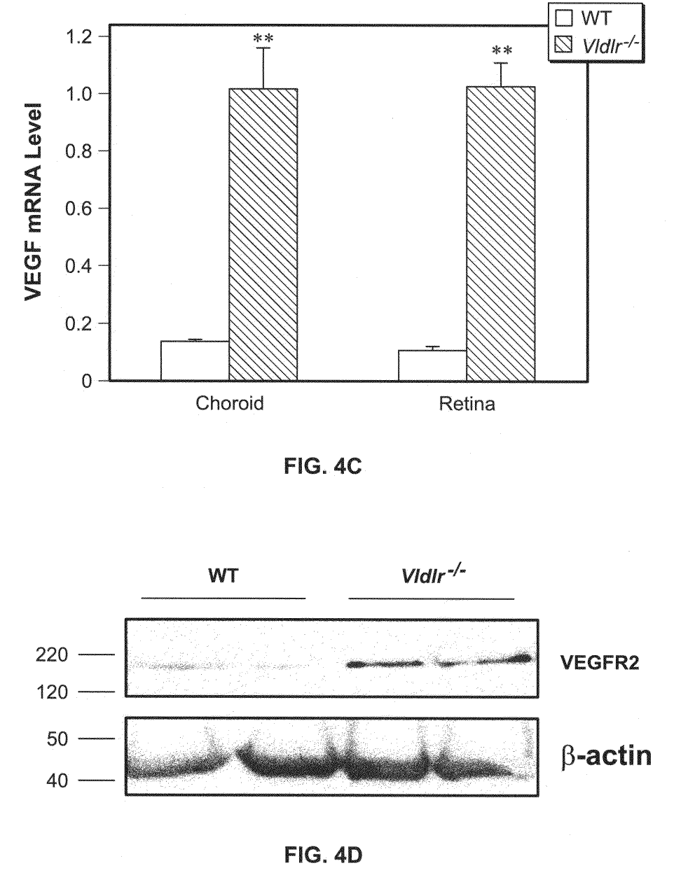

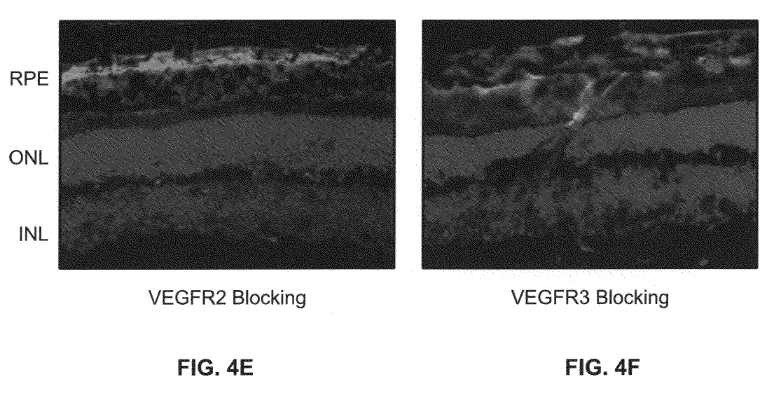

According to embodiments and as illustrated in FIGS. 4A and 4B, equal amount of eyecup proteins were separately blotted with antibodies for VEGF and for VEGFR2. The membranes were stripped and re-blotted with an anti-13-actin antibody. VEGF monomer (23 kDa) and dimer (46 kDa) were semi-quantified by densitometry, normalized by 13-actin levels and expressed as the relative ratio of wt to Vldlr−/− (mean±SD, n=3). Real-time RT-PCR showed elevated VEGF mRNA levels in the Vldlr−/− retinas and choroids (expressed as the wt to Vldlr−/− ratio, mean±SD, n=3) in FIG. 4C. In FIG. 4D, VEGFR2 levels were measured by Western blot analysis. Purified neutralizing antibodies (rat IgG) for VEGFR2 and VEGFR3 were separately injected into the subretinal space of Vldlr−/− mice at age of P12, as shown in FIGS. 4E and 4F. The injected antibodies were detected by staining with an FITC conjugated goat anti-rat antibody at P21 (green). The retinal vasculature was examined using a monoclonal anti-vWF antibody (red). Subretinal NV was attenuated by the anti-VEGFR2 antibody, but not by the anti-VEGFR3 antibody. The scale of FIGS. 4E and 4F is 10 μm.

### 4. Altered VEGF Subcellular Distribution in RPE Cells in Vldlr−/− Mice

Immunohistochemistry showed that VEGF levels were markedly increased in both Müiller glial and RPE cells of the Vldlr−/− eyes (FIG. 5). Intense VEGF signal was detected in the retinal regions displaying NV. High magnification examination of the RPE showed that VEGF signal in the wt RPE was distributed near the surface adjacent to the Bruch's membrane, consistent with previous observations of the polarized distribution of VEGF in the RPE. In contrast, some of the VEGF signals in the RPE were detected near the surface adjacent to photoreceptors in some RPE cells in Vldlr−/− mice.

As shown in FIGS. 5A and 5B and according to embodiments, ocular sections from wt (FIG. 5A) and Vldlr−/− mice (FIG. 5B) were stained with the antiVEGF antibody (green), and the nuclei counter-stained by DAPI (pseudo-colored red). As shown in the high magnification image of the boxed area in FIG. 5A, VEGF (green, e.g., arrow) was shown to be distributed near the RPE surface toward the choroid in wt mice. However, high magnification images from FIG. 5B showed that a part of VEGF is distributed near the RPE surface toward the photoreceptors and intensive VEGF signal around the neovascular region. The scale for FIGS. 5A and 5B is 20 μm, and 5 μm for the high magnification images.

### 5. Inflammation in the Vldlr−/− Retina

### Leukostasis in the Vldlr−/− Retina.

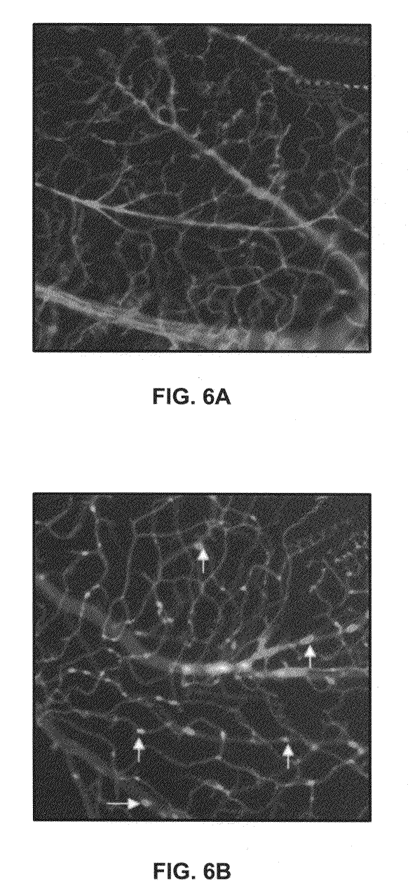

As shown by leukostasis assay, wt retina showed clear retinal vasculature−/− without detectable leukocytes adherent to the endothelium of the vasculature. In contrast, the retina of Vldlr−/− mice at the same age has multiple leukocytes adherent to the endothelium of the retinal vasculature (FIGS. 6A, 6B, and 6C). This result indicates that Vldlr−/− mice have significant leukostasis and inflammation in the retina vasculature.

According to embodiments of experimental data, retinal vascular endothelium and adherent leukocytes were stained with Con-A in 2-month old wt (FIG. 6A) and Vldlr−/− (FIG. 6B) mice and then flat-mounted. The adherent leukocytes were visualized under fluorescent microscope. Multiple leukocytes adherent to endothelium of retinal vasculature (e.g., arrows) were observed in the Vldlr−/− retina (FIG. 6B) but not in the wt retina (FIG. 6A). In FIG. 6C, adherent leukocytes were counted in the whole retinas of wt and Vldlr−/− mice (mean±SD), showing significantly increased leukostasis in Vldlr−/− retina.

### Over-Expression of Inflammatory Cytokines in the Eyecups of Vldlr−/− Mice.

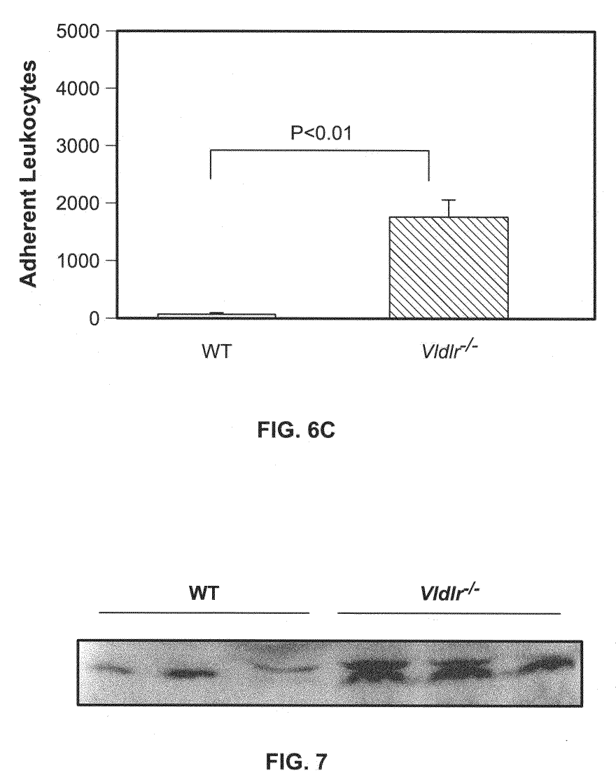

The expression of TNF-α, a major pro-inflammatory cytokine, was compared between wt and Vldlr−/− eyecups using Western blot analysis. The results showed that Vldlr−/− eyecups have significantly elevated TNF-α levels, compared to that in wt at the same age. According to embodiments of experimental data, eyecups were dissected from 2-month old wt and Vldlr−/− mice in FIG. 7. The same amount of total eyecup proteins from each mouse was loaded for Western blot analysis using an anti-TNF-α antibody. Each lane represents a sample from an individual mouse.

### Activation of NF-κB in the Eyecups of Vldlr−/− Mice.

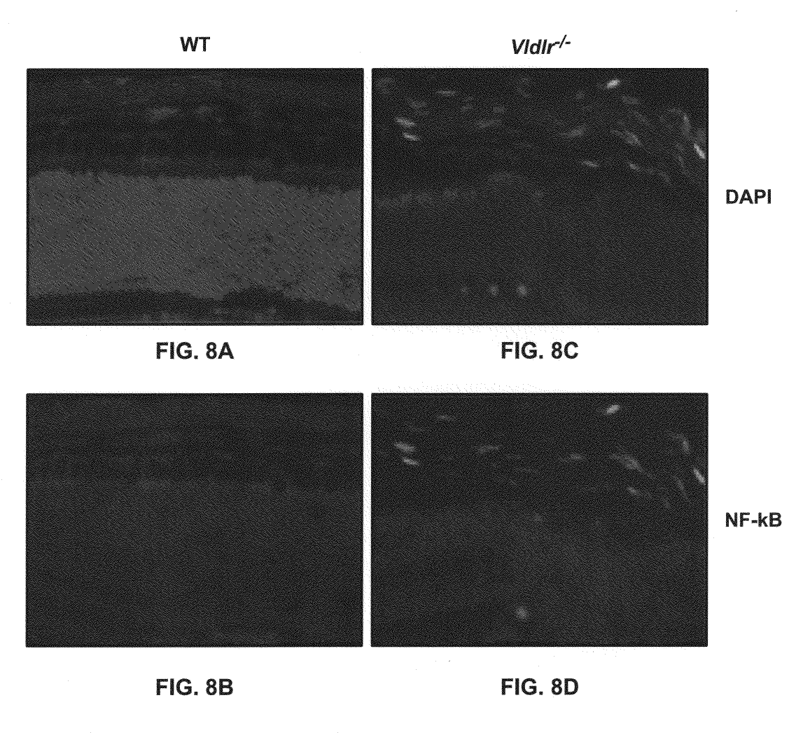

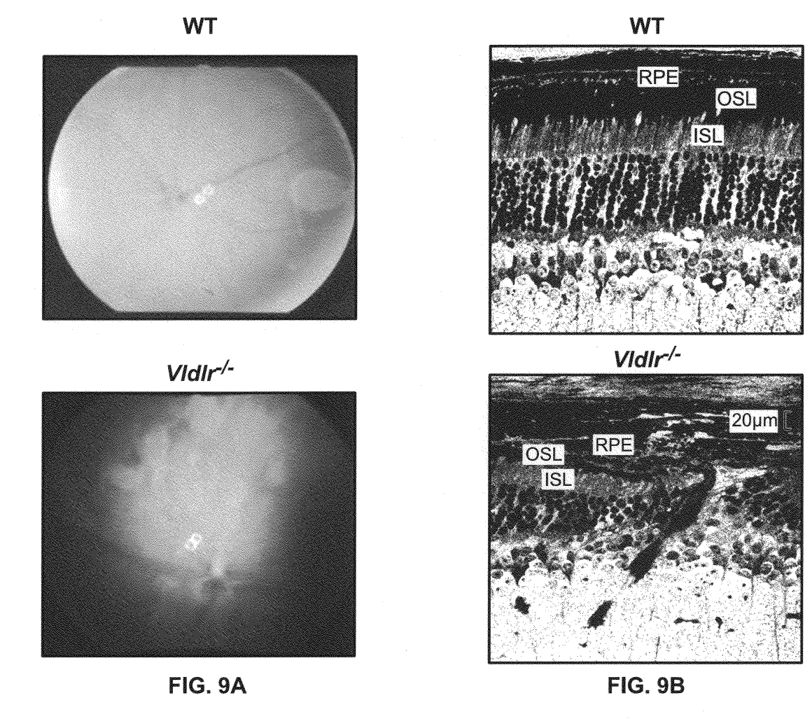

NF-κB is a key transcription factor activating inflammatory responses. Nuclear translocation is a crucial step in the NF-κB activation. Immunohistochemistry with counter-staining of the nucleus by 4′,6-diamidino-2-phenylindole (DAPI) in the eye sections showed that NF-κB levels in the nucleus were elevated in the Vldlr−/− retina and choroid, compared with that in the wt (FIGS. 8A-8D). Together with the increased pro-inflammatory cytokines and leukostasis, this result suggests that the Vldlr−/− eyecups indeed have increased inflammatory responses.

According to embodiments of experimental data, the cross eye sections from the wt (FIGS. 8A and 8B) and Vldlr−/− (FIGS. 8C and 8D) mice were stained with an anti-NF-κB antibody and the nucleus counterstained with DAPI.

### 6. Degeneration of Photoreceptors in Vldlr−/− Mice

According to embodiments of experimental data, FIG. 9A shows representative fundus images of wt and Vldlr−/− mice at age of 8 months showing hypo- and hyper-pigmentation areas in Vldlr−/− mice. In FIG. 9B, retinal plastic sections stained with Toluene blue showing shortened rod outer segments and fewer layers of nuclei in the outer nuclear layer in Vldlr−/− mice.

Retina cross sections from 8-month-old Vldlr−/− and wt mice were stained with Toluene blue. Photoreceptor outer segments were examined under light microscope. The Vldlr−/− photoreceptors showed apparently shortened outer segments, in addition to the presence of abnormal neovasculature in the photoreceptor layer (FIG. 9B). The layers of photoreceptor nuclei were also fewer than that in wt (FIG. 9B, also in FIGS. 1I and 1J). Fundus images showed that Vldlr−/− mice develop hypo- and hyper-pigmented areas (FIG. 9A), a characteristic change in the fundus images of AMD patients.

### 7. Disturbed Retinoid Visual Cycle in the Retina and RPE of Vldlr−/− Mice

In vertebrates, the visual sensing pigment is formed upon attachment of 11-cis retinal to opsin by means of a Schiff's base bond. Light stimulation causes the isomerization of 11-cis retinal to all-trans retinal, which causes disassociation from opsin, and initiation of the phototransduction cascade. In order to promote reassociation with opsin and subsequent priming for another round of phototransduction, all-trans retinal must be converted back to 11-cis retinal through a series of chemical reactions. Several enzymes in the photoreceptors and RPE are known to play a role in this process. The levels of these retinoids can be directly correlated to the level of phototransduction; therefore, the retinoid profile in the eyecups of Vldlr−/− mice was characterized.

Adult Vldlr−/− mice were dark-adapted overnight. The eyecups were dissected and homogenized in the dark. Endogenous retinoids were extracted and analyzed by HPLC as described previously. The HPLC retinoid profile showed that Vldlr−/− mice have significantly decreased amounts of 11-cis retinal and retinyl ester. In contrast, all-trans retinal levels are not significantly changed in Vldlr−/− mice. Since 11-cis retinal is the chromophore for the visual pigments, the decreased levels of 11-cis retinal further suggest compromised photoreceptor functions.

According to embodiments of experimental data, eyecups were dissected from dark-adapted wt and Vldlr−/− mice at age of 8 weeks in FIG. 10. Retinoids were extracted from the tissue homogenates and analyzed by high performance liquid chromatography (HPLC). Each form of retinoids was identified by its characteristic elution time by comparison with standards and the amounts quantified. Values are mean±SD, from 4 mice. The amounts of 11-cis retinal and total retinyl ester were significantly lower in Vldlr−/− eyecups than that in wt (P<0.001 and P<0.05, respectively).

### 8. Cone Photoreceptor Degeneration in Vldlr−/− Mice

A major feature of AMD disease pathogenesis is the specific loss of cone photoreceptors responsible for central vision and visual acuity. To examine the whether a similar phenotype exists in Vldlr−/− mice, we performed electroretinography to examine the retinal response to light stimuli. Vldlr−/− mice at 2.5 months of age were dark adapted to examine rod photoreceptor function and then light-adapted to examine cone photoreceptor function. A corneal electrode was used to measure changes in electrical potential of the eye and the amplitude of the a-wave, representing photoreceptor hyperpolarization, were quantified. The data demonstrates that there is no significant reduction in rod-derived a-wave amplitudes in Vldlr−/− mice; however, a 40% decrease was observed in cone-derived a-wave amplitudes from Vldlr−/− mice. These data further establish Vldlr−/− mice as a unique model for AMD with cone photoreceptor degeneration.

According to embodiments of experimental data, Vldlr−/− mice at 2.5 months of age were subjected to electroretinography and the A-wave amplitudes were quantified as illustrated in FIG. 11. No significant difference between Vldlr−/− mice and age matched wild-type controls were observed (p>0.05, n=4); however, a significant reduction in the cone derived A-wave was observed (p<0.05, n=4).

### 9. HIF-1α Levels are Increased in the RPE and Subretinal Space of Vldlr−/− Mice

HIF-1α is a major transcription factor activating VEGF expression. Immunohistochemistry analysis showed significantly increased HIF-1α signals in the RPE of Vldlr−/− mice, compared to the wt mice, suggesting a potential role of HIF-1α in VEGF over-expression in Vldlr−/− mice.

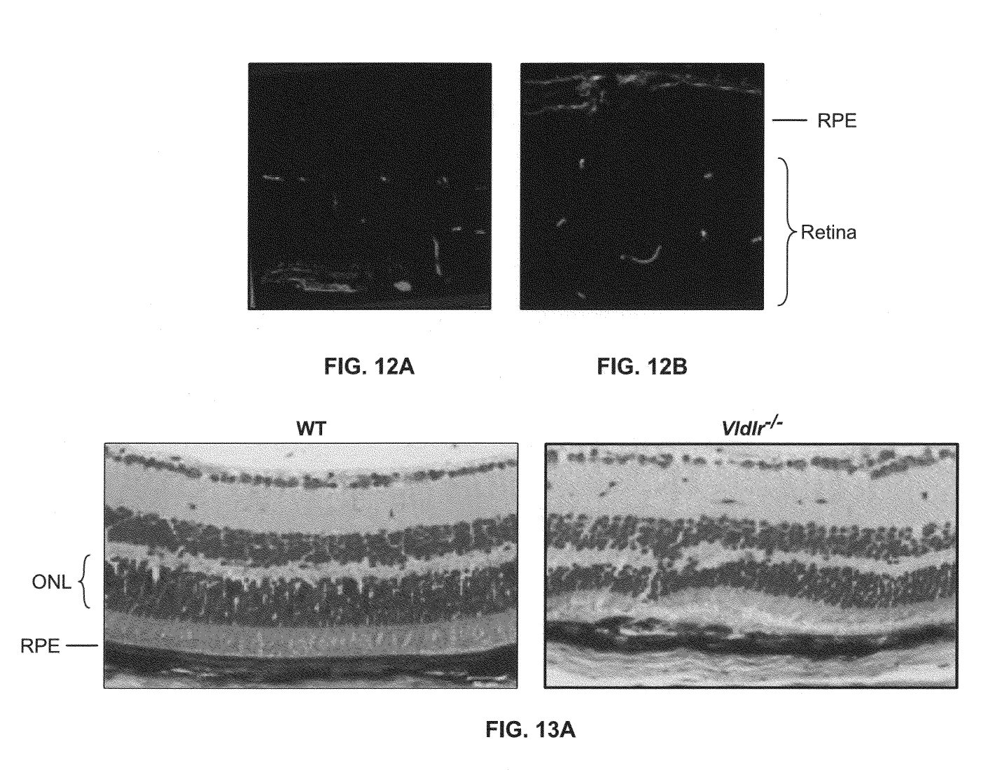

According to embodiments of experimental data, cross sections from wt (FIG. 12A) and Vldlr−/− (FIG. 12B) mouse eyes were double stained with antibodies for CD31 (endothelial marker, red) and HIF-1α (green). Elevated HIF-1α levels were observed in the RPE and subretinal space of Vldlr−/− mice. For the experiments performed in FIGS. 12A and 12B, the scale shown is 10 μm.

### 10. Progressive Photoreceptor Degeneration in Vldlr−/− Mice.

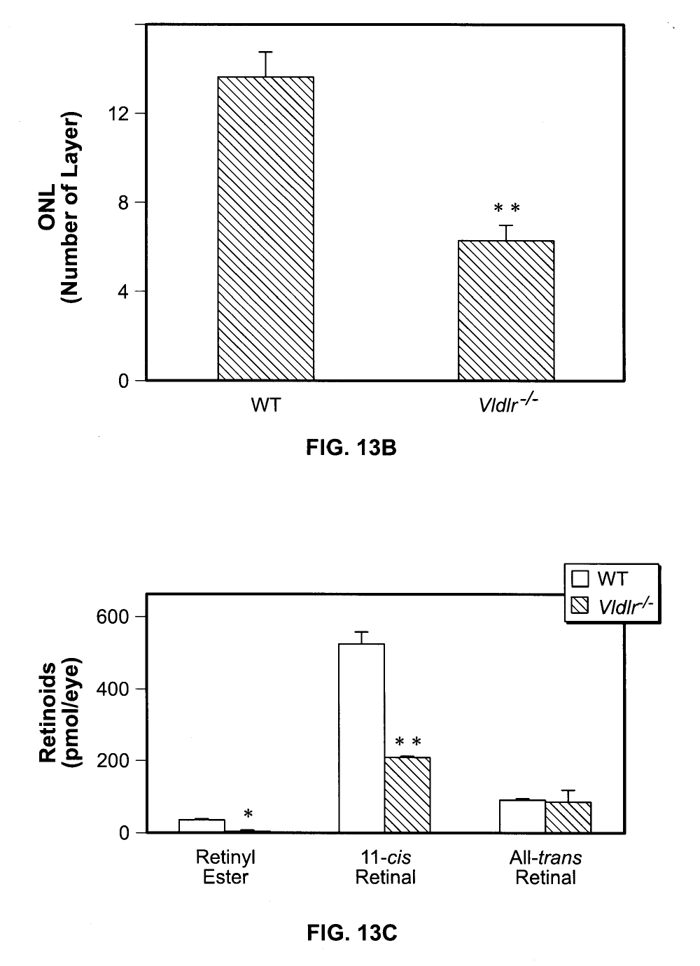

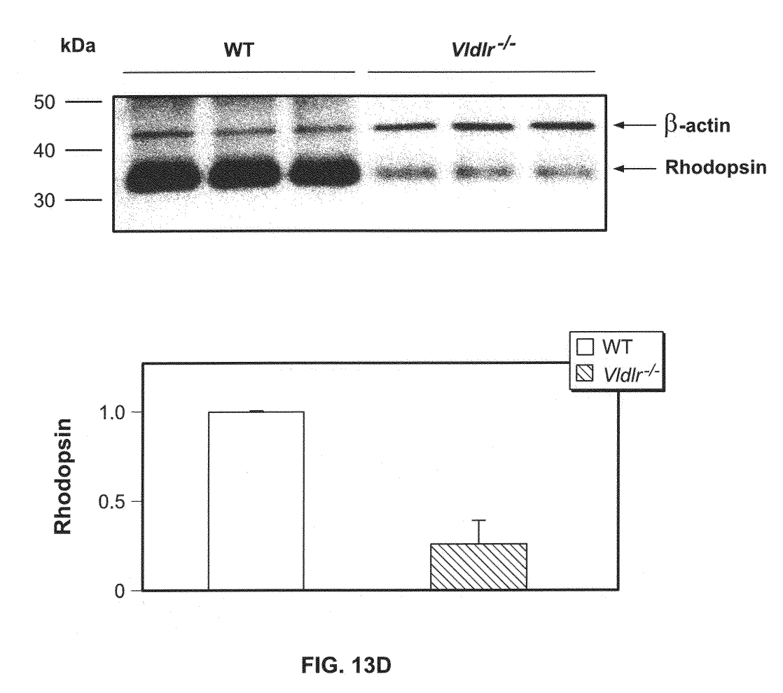

Cross retinal sections were stained with HE and layers of nucleus in the outer nuclear layer (ONL) were counted. In Vldlr−/− mice at age of 7 months, layers of nucleus in the ONL were significantly decreased compared to that of wt at the same age (FIGS. 13A and 13B). Western blot analysis of rhodopsin showed significantly decreased rhodopsin levels in Vldlr−/− retina at older ages (8 months) (FIG. 13D), further confirming retinal degeneration in this mouse model.

According to embodiments of experimental data in FIG. 13, representative crossed sections with HE staining from wt and Vldlr−/− mice at the age of 7 months showed significantly fewer nucleus layers in the ONL of Vldlr−/− mice in FIG. 13A. In FIG. 13B, nucleus layers were counted on the cross section and averaged, showing significantly decreased layers of ONL in Vldlr−/− mice (mean±SD, n=4). In FIG. 13C, retinoids were extracted from eyecups from dark-adapted wt and Vldlr−/− mice and analyzed by HPLC. Each form of retinoids was identified by its characteristic elution time by comparison with standards and the amounts quantified (mean±SD, n=4). The amounts of 11-cis retinal and total retinyl ester were significantly lower in Vldlr−/− eyecups than that in wt (P<0.001 and P<0.05, respectively). Finally, western blot analysis of rhodopsin in FIG. 13D shows equal amount of retinal proteins from 3 wt and Vldlr−/− mice at age of 8 months. Western blotting was performed with the 1D4 antibody and normalized by β-actin. The average rhodopsin levels as quantified by densitometry were significantly lower in Vldlr−/− mice (P<0.001).

### 11. Over-Expression of Inflammatory Cytokines in the Eyecups of Vldlr−/− Mice

The expression of TNF-α and COX2, major pro-inflammatory factors, was compared between wt and Vldlr−/− eyecups using Western blot analysis, and levels of soluble intercellular adhesion molecule-1 (sICAM-1) were measured by ELISA. The results showed that Vldlr−/− eyecups have significantly elevated levels of COX1, TNF-α, and sICAM-1, compared to that in wt at the same age (FIG. 15), suggesting inflammation in the Vldlr−/− retina.

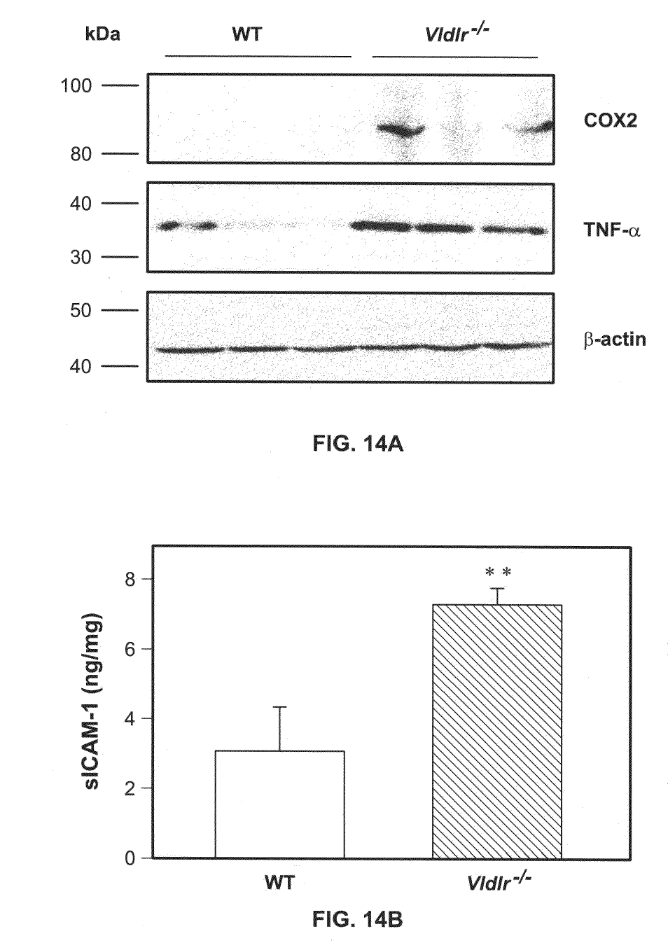

According to embodiments of experimental data and as illustrated in FIG. 14, eyecups were dissected from 2-month old wt and Vldlr−/− mice. In FIG. 14A, the same amount of total eyecup proteins from each mouse was loaded for Western blot analysis using antibodies specific for COX2 and TNF-α. The membrane was stripped and reblotted with an anti-β-actin antibody. Each lane represents an individual mouse. In FIG. 14B, soluble ICAM-1 was measured using ELISA and normalized by total protein concentrations (mean±SD, n=4).

### Materials and Methods

Animals—Animals were maintained in a 12-h light/12-h dark cycle with an ambient light intensity of 85±18 lux at the cage level. Vldlr−/− mice on the C57BL/6 background and wild-type (wt) C57BL/6 mice (The Jackson Laboratory, Bar Harbor, Me.) were used, treated and cared for in accordance with the statement for the Use of Animals in Ophthalmic and Vision Research set forth by the Association for Research in Vision and Ophthalmology. Vldlr−/− mice were genotyped following a PCR protocol recommended by The Jackson Laboratory.

Cell Culture—Human umbilical vein endothelial cells (HUVEC) were purchased from the American Type Culture Collection (Manassas, Va.). Cell culture reagents, fetal bovine serum, and chemicals were purchased from Invitrogen. ARPE19 cells were maintained in Dulbecco's modified Eagle's medium containing 3 mM L-glutamine, 10% fetal bovine serum, 100 units/ml penicillin G, and 100 μg/ml streptomycin sulfate at 37° C. in an environment containing 95% O₂ and 5% CO₂. HUVEC were cultured in endothelial cell basal medium (EBM-2, Cambrex, N.J.) maintained at 37° C. in an environment containing 95% O₂ and 5% CO₂ and supplemented with 5% fetal bovine serum, penicillin/streptomycin, and endothelial cell growth supplement (SingleQuots, Cambrex, N.J.). The cells were used in experiments between passage 4 and 6.

Fluorescein Angiography—Angiograms were performed using intracardiac injection of 10 mg/ml fluorescein isothiocyanate-conjugated high molecular weight dextran (Sigma, FD-2000S) in deeply anesthetized mice. Eyes were dissected and fixed with 4% paraformaldehyde in Hanks' balanced saline prepared immediately before use for overnight at 4° C., and retinas were flat-mounted in Fluoromount-G.

Immunohistochemistry—Eyes were enucleated and fixed in 4% paraformaldehyde overnight at 4° C. Cross-sections (5 μm) were cut and mounted on slides. To reduce autofluorescence background levels, the sections were blocked with 2% mouse serum and 10% normal goat serum in phosphate-buffered saline with 0.3% Triton X-100 for 1 h. Sections were stained with primary antibodies specific for VEGF (Santa Cruz, Calif.), β-catenin, GSK-3P, phosphorylated GSK-3β, and phosphorylated β-catenin (Cell Signaling, Danvers, Mass.), LRP5/6 (ABCAM, Cambridge, Mass.), CD31 (BD Pharmingen), and a rabbit anti-RDH10 antibody. Retinal sections were incubated with the primary antibodies for 1 h and washed thoroughly with phosphate-buffered saline. Secondary antibodies were added and incubated with the sections for 1 h. The sections were finally washed in phosphate-buffered saline and mounted in Fluoromount-G.

VEGF ELISA—The human VEGF QuantiGlo ELISA kit (R&D Systems, Inc., Minneapolis, Minn.) was used to measure VEGF levels in HUVEC and ARPE19 cells, and the mouse VEGF Quantikine ELISA kit (R&D Systems, Inc.) was used for mouse tissues following the manufacturer's protocol. The samples of the culture medium were concentrated 10 times, and the samples from mouse tissues were diluted 10 times to ensure that the VEGF concentration fell within the range of the VEGF standard curves.

Western Blot Analysis—The same amount (10 μg) of total proteins from mouse eyecups were used for Western blot analysis using specific primary antibodies for each protein and blotted with an horseradish peroxidase-conjugated secondary antibody. The signal was developed with a chemiluminescence detection kit (ECL; Amersham Biosciences). Blots were then stripped and re-blotted with an antibody specific for μ-actin. Each protein band was semiquantified by densitometry and normalized by μ-actin levels in the same gel.

Quantitative Real-time Reverse Transcription (RT)-PCR—Mice eyes were enucleated immediately after death into chilled diethylpyrocarbonate-treated normal saline, and the retinas were dissected. Total RNA was isolated using a commercial kit (Qiagen, Santa Clarita, Calif.). Primers (VEGF forward and VEGF reverse) were designed from the cDNA sequences spanning >1-kb introns using the Primer3 software. Total RNA (1.0 μg) was used for RT reactions, and 1 μl of the RT product and 3 pmol of primers were used for real-time PCR with a SYBR Green PCR Master Mix (Applied Biosystems). Fluorescence changes were monitored after each cycle. Amplicon size and reaction specificity were confirmed by 2.5% agarose gel electrophoresis. All reactions were performed in triplicate. The average C_{T} (threshold cycle) of fluorescence unit was used to analyze the mRNA levels. The VEGF mRNA levels were normalized by 18 S ribosomal RNA levels. Quantification was calculated as follows: mRNA levels (percent of control)=2Δ(ΔC_{T}), with ΔC_{T}=C_{T, VEGF}−C_{T, 18 S}, and Δ(ΔCT)=ΔC_{T,wt sample}−ΔC_{T,Vldlr}−/− sample.

Injection of Neutralizing Antibodies Specific for VEGFR2 and VEGFR3—Purified neutralizing antibodies for VEGFR2 and VEGFR3 (generous gifts from ImClone System) were separately injected into the subretinal space of Vldlr−/− mice at age of P12. The eyes were dissected at P21 and fixed for NV analysis.

Transfection of Small Interference RNA (siRNA)—The Cy3-labeled siRNA targeting VLDLR was commercially purchased from Ambion (Austin, Tex.). Transfection was performed using siPORT Amine (Ambion) following the instructions of the manufacturer. Briefly, 5×10⁶ HUVEC were incubated with the transfection mixtures containing 100 pmol of the Cy3-labeled siRNA for VLDLR or a Cy3-labeled control siRNA with a scrambled sequence for 24 h at 37° C. in 5% CO₂. Twelve hours after the transfection, the cells were washed twice with phosphate-buffered saline to remove transfection mixtures and cultured in Dulbecco's modified Eagle's medium containing 5% fetal bovine serum until they were used.

Subretinal Injection of Purified Mouse Dickkopf-1 (DKK1) Protein—Purified DKK1 (R&D System, Minnesota) was injected into the subretinal space of the right eye (5 μg/eye), and the same amount of bovine serum albumin (BSA) was injected into the left eye of Vldlr−/− mice at age of 4 weeks. Eyeballs were harvested 24 h after the injection, and the eyecups were dissected for analysis.

Intravitreal injection: All solutions will be sterilized by filtration and assessed for endotoxin. Animals will be anesthetized, and compounds will be injected into the vitreous of the one eye through the pars plana using a Hamilton syringe. The left eye will receive the same volume of vehicle and will be used as the control. Following injection, the animals will receive equal amounts of topical antibiotic ointment on both eyes. The animals will then be kept in normoxic conditions until the necessary time point for evaluation

Measurement of vascular permeability: Vascular permeability will be quantified by measuring FITC-BSA leakage from blood vessels into the retina following a method with modifications. The mice anesthetized and FITC-BSA (10 mg/kg body weight) is injected through the femoral vein under microscopic inspection. After injection, the mice are kept on a warm pad for 3 h to ensure the complete circulation of FITC-BSA. Then the chest cavity is opened, blood is collected through right atrium. Mice are perfused via the left ventricle to remove unbound dye with 1× PBS (pH 7.4), which is pre-warmed to 37° C. to prevent vasoconstriction. Immediately after perfusion, the eyes are enucleated, and the retinas are carefully dissected under an operating microscope. The fluorescein-albumin is extracted by sonication and centrifugation. The fluoresce density of fluorescein-albumin from supernatant and serum is measured at excitation wave 485 nm/emission wave 530 nm. Retinal protein levels are measured in the pellet by Bradford assays with quantification at A280. The FITC-BSA levels in the retina are then calculated by the supernatant fluoresce density and normalized to retinal protein levels and normalized to serum FITC levels.

Statistical Analyses: All assays utilizing quantification will be subjected to rigorous statistical testing. Significance will be determined using one-way analysis of variance and the appropriate post-hoc tests using Bonferroni's pairwise comparisons (Prism, version 3.02; GraphPad).

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
identifying a candidate therapeutic agent for the treatment of an eye-related diseased characterized by at least one of vascular leakage and inflammation;
causing the agent to be administered to a mouse whose cells comprise at least a disrupted very low-density lipoprotein receptor, the disruption being sufficient to disrupt substantial expression of very low-density lipoprotein receptor;
causing a determination of the effectiveness of the agent in treating the eye-related disease;
wherein the mouse exhibits choroidal neovascularization; and
wherein the mouse exhibits at least one of neo-vascularization in the eye and inflammation of the eye.

2. The method of claim 1, wherein the eye-related disease is age-related macular degeneration.

3. The method of claim 1, wherein the eye-related disease comprises over-active wnt pathway signaling.

4. The method of claim 1, wherein the eye-related disease comprises overexpression of LRP5 or LRP6n.

5. The method of claim 1, wherein the agent comprises at least anti-angiogenic compounds.

6. The method of claim 1, wherein the agent comprises at least anti-inflamatory compounds.

7. The method of claim 1, wherein the agent comprises at least anti-vascular permeability compounds.

8. The method of claim 1, wherein the agent comprises at least compounds preventing photoreceptor degeneration.

9. A method comprising:
identifying a candidate therapeutic agent for the treatment of age-related macular degeneration;
causing the agent to be administered to a mouse whose cells comprise at least a disrupted very low-density lipoprotein receptor, the disruption being sufficient to disrupt substantial expression of very low-density lipoprotein receptor;
causing a determination of the effectiveness of the agent in treating the age-related macular degeneration;
wherein the mouse exhibits choroidal neovascularization; and
wherein the mouse exhibits at least one of neo-vascularization in the eye and inflammation of the eye.

10. The method of claim 9, wherein the agent comprises at least anti-angiogenic compounds.

11. The method of claim 9, wherein the agent comprises at least anti-inflamatory compounds.

12. The method of claim 9, wherein the agent comprises at least anti-vascular permeability compounds.

13. The method of claim 9, wherein the agent comprises at least compounds preventing photoreceptor degeneration.
